(19) United States
(12) Patent Application Publication
(10) Pub. No.: US 2009/0132003 A1
(43) Pub. Date: May 21, 2009

(54) Wireless Electrical Stimulation of Neural Injury

(75) Inventors: Richard B. Borgens, Delphi, IN (US); Pedro Irazoqui, Lafayatte, IN (US)

Correspondence Address: David B Quick;ICE MILLER, Suite 3100, One American Square, Indianapolis, IN, 46282-0200, US

(73) Assignee: PURDUE RESEARCH FOUNDATION, West Lafayette, IN (US)

(21) Appl. No.: 12/083,791

(22) PCT Filed: Oct. 20, 2006
(86) PCT No.: PCT/US2006/041089

§ 371 Date: Apr. 18, 2008

Related U.S. Application Data
(60) Provisional application No. 60/729,248, filed on Oct. 21, 2005.

(51) Int. Cl. A61N 1/36

(52) U.S. Cl. 607/50

(57) ABSTRACT

An apparatus for wireless electrical stimulation of a neural injury includes a first electronic implant (102) is configured to generate a first potential difference relative to a body of a patient and a second electronic implant (104) configured to generate a second potential difference relative to the body of the patient. The second potential has a polarity opposite the polarity of the first potential difference. The second electronic implant (104) is configured to be wirelessly communicatively coupled and electrically coupled to the first electronic implant (102) when spaced apart therefrom in the body of a patient.

### BACKGROUND

Injury to the spinal cord or central nervous system can be one of the most devastating and disabling injuries possible. Depending upon the severity of the injury, paralysis of varying degrees can result. Paraplegia and quadriplegia often result from severe injury to the spinal cord. The resulting effect on the sufferer, be it man or animal, is severe. The sufferer can be reduced to a state of near immobility or worse. For humans, the mental trauma induced by such severe physical disability can be even more devastating than the physical disability itself.

When the spinal cord of a mammal is injured, connections between nerves in the spinal cord are broken. The injured portion of the spinal cord is termed a “lesion.” Such lesions block the flow of nerve impulses for the nerve tracts affected by the lesion with resulting impairment to both sensory and motor function.

To restore the lost sensory and motor functions, the affected motor and sensory axons of the injured nerves must regenerate, that is, grow back. Unfortunately, any spontaneous regeneration of injured nerves in the central nervous system of mammals has been found to occur, if at all, only within a very short period immediately after the injury occurs. After this short period expires, such nerves have not been found to regenerate further spontaneously.

Studies have shown, however, that the application of a DC electrical field across a lesion in the spinal cord of mammals, can promote axon growth, and the axons will grow back around the lesion. Since the spinal cord is rarely severed completely when injured, the axons need not actually grow across the lesion but can circumnavigate the lesion through remaining spinal cord parenchyma.

For optimal results in a human patient, a uniform electrical field of a desired strength is imposed over about 10 cm to 20 cm of damaged spinal cord for a beneficial clinical outcome. Ideally, this uniform field is imposed across the entire cross section of the spinal cord over this longitudinal extent, because of the general segregation of descending (motor) tracts to the ventral (anterior) cord, and the segregation of important (largely sensory) tracts to the posterior (dorsal) spinal cord. In paraplegic canines, this electrical field has been directly measured (Richard B. Borgens, James P. Toombs, Andrew R. Blight, Michael E. McGinnis, Michael S. Bauer, William R. Widmer, and James R. Cook Jr., Effects of Applied Electric Fields on Clinical Cases of Complete Paraplegia in Dogs, J. Restorative Neurology and Neurosci., 1993, pp. 5:305-322). In man however, the cross sectional area of the spinal cord is approximately two to four times that of the small to medium sized dogs treated in clinical trials, and actual invasive measurement of the imposed electrical fields in response is not feasible on human patients.

Based on the responses of human paraplegics and quadriplegics to prior art therapies involving the application of an oscillating DC electrical field across a lesion in the spinal cord using three pairs of electrodes, it appears that the dorsal (posterior) location of three pairs of electrodes did not produce a uniform field over the entire unit area of the patient's spinal cord. This was revealed by the domination of sensory recovery in these patients (˜thirtyfold over historical controls) compared to motor recovery (˜twofold greater than historical controls) using the ASIA scoring system. Thus, the voltage gradient was highest nearest to the actual placement of two pairs of electrodes on either side (two tethered to the right and left lateral facets) and the third pair sutured to the paravertebral muscle and fascia of the dorsal (posterior) facet-rostra and caudal of the spinal cord lesion (Shapiro, et al., Oscillating Field Stimulation for Complete Spinal Cord Injury in Humans: a Phase 1 Trial, Journal of Neurosurg. Spine 2, 2005, pp. 3-10).

It would be desirable to provide a device to generate a DC electrical field across the spinal cord lesion of a human in order to facilitate the creation of a uniform electrical field over the affected area. It would be further desirable to provide a method for implanting pairs of discrete electrodes that communicate with each other and an external controller to facilitate the creation of an adjustable uniform electrical field over the affected area of the injured spinal cord.

### SUMMARY

According to one aspect of the disclosure, an apparatus for wireless electrical stimulation of a neural injury includes a first and second electronic implant. The first electronic implant is configured to generate a first potential difference relative to a body of a patient and the second electronic implant is configured to generate a second potential difference relative to the body of the patient. The second potential has a polarity opposite the polarity of the first potential. Both electronic implants are configured to communicate wirelessly with each other within the body of a patient, and with an external controller from within the body of a patient. The first electronic implant and second electronic implant are configured to change their polarities substantially simultaneously.

According to one aspect of the disclosure, an apparatus for stimulating axon growth of the nerve cells in the spinal cord of mammals, comprises a first electronic implant having an electrode, a voltage generating circuit to create a voltage potential difference between the electrode and the mammal, and a polarity reversing circuit electrically coupled to the voltage generating circuit and configured to reverse the polarity of the voltage potential difference between the electrode and the body of the mammal each time a predetermined period of time elapses and a second electronic implant having an electrode, a voltage generating circuit to create a voltage potential difference between the electrode and the mammal, and a polarity reversing circuit electrically coupled to the voltage generating circuit and configured to reverse the polarity of the voltage potential difference between the electrode and the body of the mammal each time a predetermined period of time elapses, the second electronic implant being communicatively coupled to first electronic implant when spaced apart therefrom, wherein said first electronic implant and said second electronic implant are configured to change their polarities substantially simultaneously.

Additional features and advantages of the invention will become apparent to those skilled in the art upon consideration of the following detailed description of a preferred embodiment exemplifying the best mode of carrying out the invention as presently perceived.

### BRIEF DESCRIPTION OF THE DRAWINGS

The features and advantages of this invention, and the methods of obtaining them, will be more apparent and better understood by reference to the following descriptions of embodiments of the invention, taken in conjunction with the accompanying drawings, wherein:

FIG. 1 shows a block diagram of a neural injury treatment device including an external device and two discrete electrodes capable of generating a controllable potential difference between the electrodes;

FIG. 2 a view of a capacitive electrode with parts broken away and internal components represented diagrammatically;

FIG. 3 is a view of a capacitive electrode of FIG. 1 received in the hollow lumen of a wide bore trochanter for implantation into the body of a patient suffering neural damage in a minimally invasive manner;

FIG. 4 shows a schematic of a circuit for generating an oscillating electrical field for stimulating nerve regeneration;

FIG. 5 shows a schematic of a voltage controlled oscillator of the circuit of FIG. 4;

FIG. 6 shows a schematic of an electromagnetic power coupling portion of the circuit of FIG. 4;

FIGS. 7A-B shows a schematic of a biphasic pulse generator that may serve as the multi-phasic pulse generator of the circuit of FIG. 4;

FIG. 8 is a wave diagram of a triphasic pulse;

FIG. 9 is a block diagram of a triphasic pulse generator that that may serve as the multi-phasic pulse generator of the circuit of FIG. 4;

FIG. 10 shows a graph that portrays the effect of an applied steady DC field over time on the growth of cathodal and anodal facing axons;

FIG. 11 shows a graph that portrays the effect of an applied oscillating field over time on the growth of cathodal and anodal facing axons; and,

FIG. 12 shows a graph that portrays the effect of an applied pulse wave modulated oscillating field over time on the growth of cathodal and anodal facing axons.

### DESCRIPTION

For the purposes of promoting an understanding of the principles of the disclosure, reference will now be made to the embodiments illustrated in the drawings and described in the following written specification. It is understood that no limitation to the scope of the disclosure is thereby intended. It is further understood that the present invention includes any alterations and modifications to the illustrated embodiments and includes further applications of the principles of the disclosure as would normally occur to one skilled in the art to which this invention pertains.

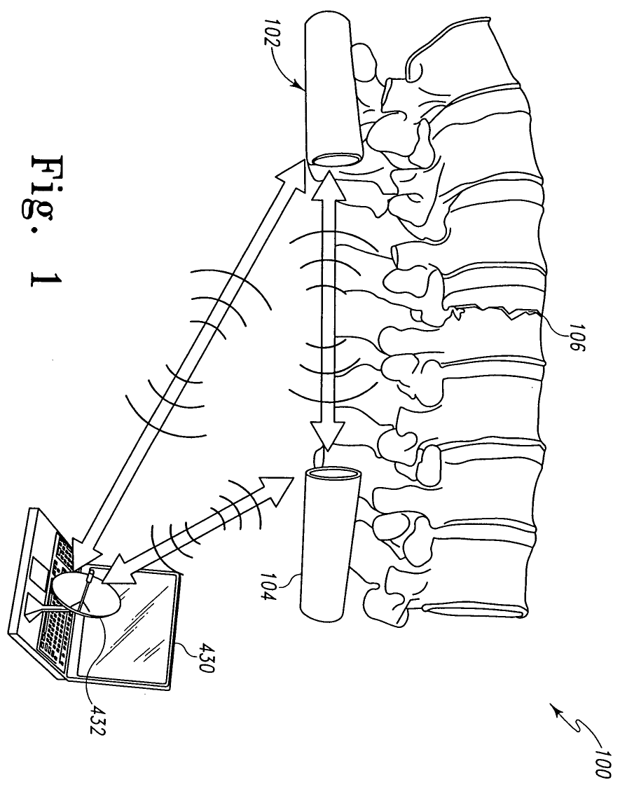

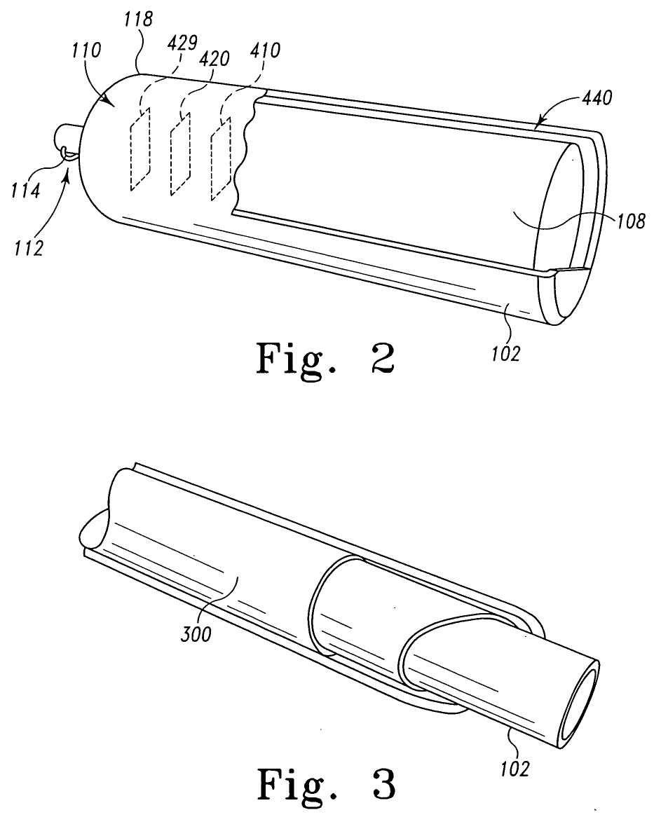

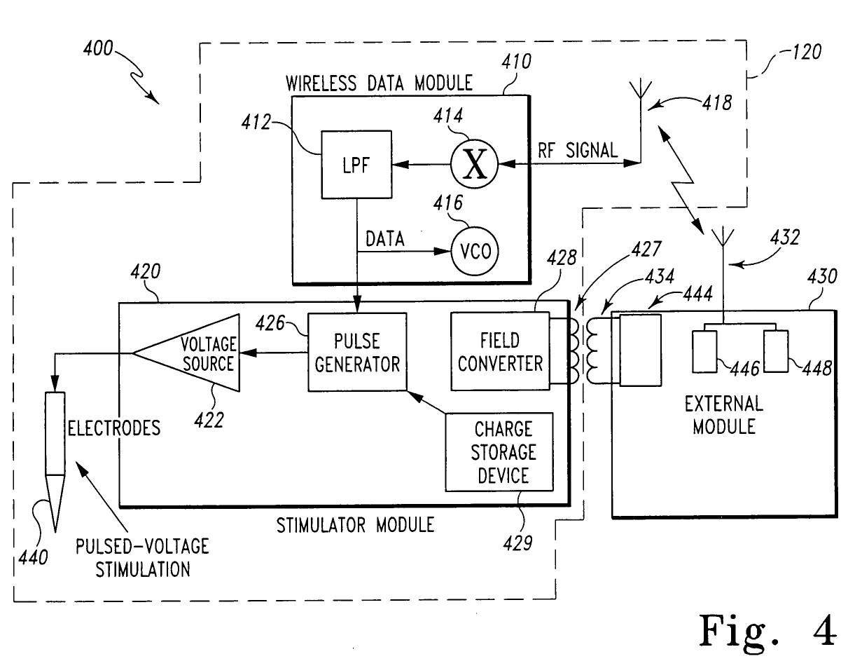

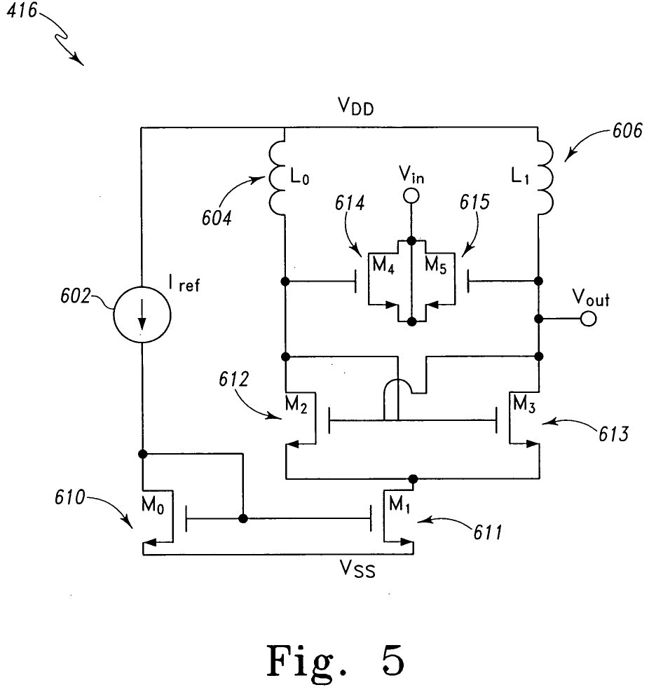

As shown, for example, in FIGS. 1-4, a neural injury treatment device 100 includes two electronic implants 102, 104 and an external module 430. Each electronic implant 102, 104 includes a skin 110 forming a case or enclosure 118 and internal electronics 120. The skin 110 may comprise a ceramic and/or titanium, making the case 118 of the electronic implants 102, 104 of the neural injury treatment device 100, in theory, surgically implantable for the life of the patient. The skin 110 provides an enclosure 118 for the internal electronics 120 of the electronic implants 102, 104 of the neural injury treatment device 100. Thus, it is within the scope of the disclosure for the enclosure 118 formed by skin 110 to be fabricated from other bio-compatible materials, alone or in combination, that form an enclosure 118 that provides sufficient protection to the enclosed internal electronics 120 of the electronic implants 102, 104, and includes a portion that is transparent, substantially transparent or translucent to electromagnet fields and radiation. In one specific embodiment, the skin 110 comprises a case 118 fabricated from medically approved ceramic available from the Sigma-Aldrich Corporation. In another embodiment, the skin 110 comprise a Titanium case.

One advantage of the enclosure 118 being fabricated from skin 110 comprising ceramic is that lifetime implantable ceramic cases may be formed that provide the ability to mold the case 118 into a desired shape. Additionally, because ceramic is transparent, substantially transparent or translucent to electromagnetic radiation, it may be desirable to fabricate at least a “window” of the case 118 from skin 110 comprising ceramic in order to facilitate the transmission of electromagnetic waves carrying power and data. The ceramic material used to fabricate the skin 110 may be obtained as a powder to facilitate the custom molding of shapes. For example, one useful shape for the case 118 fabricated from the skin 110 may be to mold it into a container having a cylindrical outer shape, as shown, for example, in FIGS. 1-3. In one embodiment the cylindrical case 118 formed by the skin 110 of each electronic implant 102, 104 is 1-2 mm in diameter and 10-12 mm in length. Because ceramic is transparent to electromagnetic waves, such a skin 110 facilitates the functionality of telemetry, antennae 418, fail-safe off, and other capabilities associated with telemetry.

Each electronic implant 102, 104 of the neural injury treatment device 100 includes internal electronics 120 received within the case 118 formed by the skin 110. The internal electronics 120 may include a wireless data module 410, a stimulator module 420, a charge storage device 429, and a capacitive electrode 440 according to one embodiment of the disclosed device. The charge storage device 429 may be a transcutaneous rechargeable battery, a capacitor, an inductive charging coil, or the like.

The external module 430 of the neural injury treatment device 100 may include data acquisition 446, device programming 448, and inductive power-coupling hardware or subcutaneous charging device 444 configured to interface with the wireless data module 410 and the stimulator module 420 of the implant 102, 104 which together form a fully implantable stimulator system.

The described, fully implantable, stimulator system provides a new means to treat Spinal Cord Injury (“SCI”); though it is not limited to this, as other Nerve Injuries will likely benefit as well from being treated using the disclosed neural injury treatment device 100. The basis of the therapy is the proven ability of imposed gradients of DC voltage (˜200-900 μV/mm) to induce functional regeneration/reconnection of mechanically injured spinal axons in fish, rodent, dog, and human beings. To date all means to achieve geometrically precise orientation, together with a significant magnitude, of a gradient of voltage imposed over soft tissues of the body are invasive. Non-invasive (transcutaneous) imposition of electric fields (as performed in some orthopedic therapies) is not possible with DC applications (the former are relatively high frequency AC applications). To attempt a similar application using DC would require excessive driving voltages, would produce insufficient field strengths (EMF supported, Hall Effect Voltages) in the tissues, and an inability to focus/orient the imposed voltage gradient to the geometries of the relevant nerve tracts.

The existing technology used to treat SCI utilizes an implanted voltage source to power a current regulated DC electrical field (˜600 μV/mm; 15 min duty cycle of polarity) imposed over the white matter of the damaged cord. The long axis of the field is parallel with the long axis of the spinal cord—this geometry is exploited since nerve fibers are known to be induced to grow towards the negative pole (cathode of the imposed voltage). They are known to retract from the positive pole (anode). Long tract bundles of nerve fibers are parallel and aligned—running in the rostral/caudal direction in mammalian spinal cords. The duty cycle is as important as the geometry to achieve a useful clinical outcome, i.e. nerve fiber regeneration within the spinal cord white matter (containing only nerve fibers or axons) is initiated towards the brain (ascending tracts) and also towards the body (descending tracts) by reversal of the polarity of the electric field approximately every 15 minutes.

Present and near term technology is very limiting since arrays of stimulating electrodes (up to 4 pair; standard Teflon-insulted platinum/iridium pacemaker cable) are surgically located rostrally and caudally of the spinal cord lesion 106. The units now used in human spinal cord injury are surgically implanted under general anesthesia between one to three weeks after the first operation (decompressive surgery and spinal stabilization in the trauma center). The stimulator and electrodes are later removed surgically—again under general anesthesia—in a third operation generally fourteen weeks later. Because the electric field is associated with current flow, wires must be used to complete the circuit imposed on the spinal cord tissues. The neural injury treatment device 100 described herein permits minimal surgery—likely utilizing only local anesthesia—due to miniaturization of the electronics, and the manner in which the medically efficacious field is applied without the use of wire electrodes.

The disclosed device 100 generates a pulsed DC electric field of programmable character in magnitude, latency, rise time, duration, and reversal of polarity (duty cycle) between the electronic implants 102, 104 which are spaced apart following implantation, as shown, for example, in FIG. 1. In one embodiment of the device 100, each electronic implant 102, 104 is in telemetric communication with the other electronic implant 104, 102 and the external module 430 thereby creating a three-way telemetry device. In one presently preferred embodiment of device 100, each disclosed electronic implant 102, 104 includes micro-engineered ASIC circuitry implementing the data module 410 and stimulator module 420 as well as the antenna 418. Each disclosed electronic implant 102, 104 also includes a transcutaneously rechargeable voltage source 429.

Since the inception of the use of imposed gradients of DC voltage for the treatment of nerve injuries, all known prior applications of the treatment involved an imposed gradient of voltage (“electrical field” or “E Field”) produced by a stimulator system driving current through the resistance of body tissues. Recent understanding of the mechanisms of galvanotaxis in cells and the initiation of growth responses in neurites reveal that a significant part of the response is governed by the imposed voltage that is not completely dependent on current flow (i.e. voltage-mediated, not current-mediated mechanisms of action).

For example, initiation of apical growth in cells towards the cathode is dependent on the upregulation of receptors and receptor complexes that are initially homogeneously distributed within the plane of the cell membrane. Based on their charge and their association with the aqueous phase of the membrane, these receptors are moved and sequestered to one specific locale of the cell by the processes of lateral electrophoresis (former) and electroosmosis (latter). This asymmetrical distribution, according to the currently accepted understanding of the process, induces growth in the direction predicted by the accumulation of receptors for specific substrate preferences (such as N-CAMS, fibronectin, laminen, collagin etc.), and soluble growth factors (such as neurotrophic and neurotropic molecules). Thus a voltage difference across the expanse of the cell is sufficient to induce lateral electrophoresis/asymmetrical receptor distribution—which results in oriented growth.

The disclosed device is configured to take advantage of the recognition that the above described cell response can be accomplished by using a voltage difference not associated with current flow (so-called capacitative potential drops). For example a substantial difference in voltage is expressed between the plates of a capacitor, but without current flow across the air (or other dielectric) gap. It is also known that if two metal electrodes are placed into a container of conductive solution in series with a battery-electric (ionic) current will flow between the electrodes associated with a three-dimensional electrical field produced in the aqueous media. If one of these electrodes is then insulated and returned to the media, a voltage difference will still exist between the pair. The disclosed device 100 utilizes this known phenomenon to facilitate treatment of neural injuries utilizing an applied electric field across the injury site.

The disclosed neural injury treatment device 100 advantageously exploits the above by utilizing electronic implants 102, 104. Each of these electronic implants 102, 104 is a complete Wireless Electronic Stimulator. Each of these electronic implants 102, 104 is actually a miniature stimulator 420 and electrode 440. In one embodiment the body 108 of each electronic implant 102, 104 is metal (titanium) and serves as an active electrode 440. In such embodiment, the body 108 acting as electrode 440 is coated in a biostable ceramic “skin” 110 to form the case 118. A naked metallic ground electrode 112 extends from one end of the cylindrical case 118. This extension 112 contains suture tabs 114 and is designed to be firmly sutured to soft tissues to produce a stable electrical “ground” connection with the body tissues. Each electronic implant 102, 104 contains a transcutaneously rechargeable power source 429, operational circuitry, and telemetry. The miniaturization of these electronic implants 102, 104 packages is facilitated by advancements in micro and nano fabrication technology and manufacturing.

The 3-way telemetry allows “wireless” imposition of a voltage gradient setting the character of the pulsatile or steady capacitive electric field. Monitoring the fields real time and “past-time” parameters is accomplished by micro-telemetry. Moreover, each of the two WES electronic implants 102, 104 is also in communication with the other (producing three way telemetry) such that when one electronic implant 102, 104 of the pair functions as an anode, the other electronic implant 104, 102 functions as a cathode.

This is most easily achieved by externally controlling the pair in a “master” and “slave” configuration, which requires that only one of the pair of WES electronic implants 102, 104 is actively modulated by the external control unit 430. Whatever the polarity of the “master” WES electronic implant 102, 104, telemetry between the pair sets the other 104, 102 to be a default of opposite polarity. This permits the “oscillation” of the DC voltage produced between the two WES electronic implants 102, 104. The character of stimulation, and the reversal of polarity of the electric field, may be set by the clinician at a chosen clinically effective duty cycle (typically every 15 minutes.

In one embodiment, if one WES electronic implant 102, 104 is located rostrally to the injury site or lesion 106 by two vertebral segments, the other electronic implant 104, 102 is located caudally equidistant from the lesion 106, as shown, for example, in FIG. 1. The voltage drop between the electronic implants 102, 104 will be imposed such that the long axis of the electric field will be parallel to the cord and the nerve tracts within it. This is the preferred arrangement to stimulate long tract nerve growth in both directions, i.e. towards and away from the brain.

Surgical location of the WES electronic implants 102, 104 is facilitated by their very small diameter (1-2 mm) and cylindrical shape. Insertion into the body can be performed with a wide bore trochanter 300—so that each WES electronic implant 102, 104 is ejected into place using a fiber optic as used in many conventional orthoscopic surgeries. This usually requires only “local” anesthesias. The electronic implants 102, 104 may be held in place with a vibration-sensitive conductive adhesive. The latter, like light sensitive dental adhesives, is hardened by exposure to an externally applied vibration of a particular frequency. This secures the WES electronic implants 102, 104 in place within soft tissues until connective tissue formation envelopes and immobilizes the implants 102, 104. Conductive adhesives also produce a good electrical connection between soft tissues and the metallic extension “ground” electrode 112. Often the electronic implants 102, 104 of the WES system 100 may likely be implanted at the time of decompressive surgery. If so, only a modest alteration in surgical approach would be required to suture the ground electrode 112 of each electronic implant 102, 104 to soft connective tissues—without the use of the adhesive—at the discretion of the surgeon.

FIG. 4 shows a schematic of the circuit 400 for generating an oscillating electrical field for stimulating nerve regeneration. The circuit 400 provides a means to treat spinal cord injury, as well as peripheral nerve injuries. The circuit 400 facilitates these treatments by providing imposed gradients of DC voltage between about 200 to about 900 μV/mm. These voltage gradients may induce functional regeneration and reconnection of mechanically injured neural axons in vertebrates.

The circuit 400 may include the wireless data module 410, the stimulator module 420, the external module 430, and the electrodes 440. The wireless data module 410 may include a low-pass filter 412, a transceiver 414, a voltage controlled oscillator 416, and an antenna 418. The low-pass filter 412 may be an active amplifier with low-frequency cutoff. The low-pass filter 412 may also include or be comprised of on-chip or off-chip passive resistive and capacitive devices. The transceiver 414 may be a mixer. The voltage controlled oscillator 416 may be a cross-coupled high-frequency oscillator. The antenna 418 may be a planar microstrip antenna or a monolithic microwave integrated-circuit (MMIC) radiating structure integrated with or bonded to the application specific integrated circuit. The components of the circuit 400 may be CMOS or BiCMOS. Preferably, to minimize the size of the electronic implants 102, 104, the internal electronics 120 of circuit 400 are fabricated utilizing microelectronic fabrication techniques. The internal electronics 120 of circuit 400 may be fabricated on an application specific integrated circuit (“ASIC”).

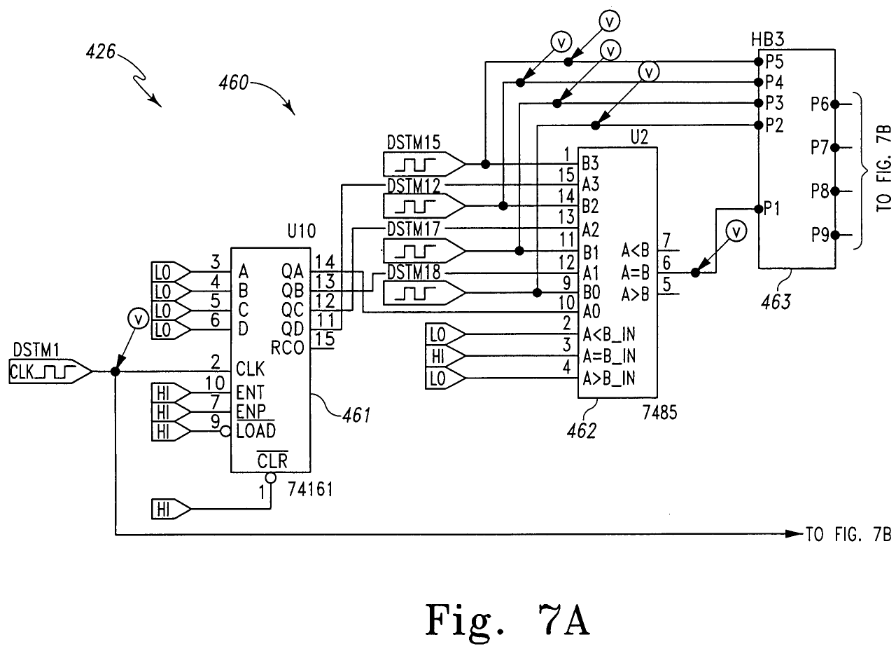

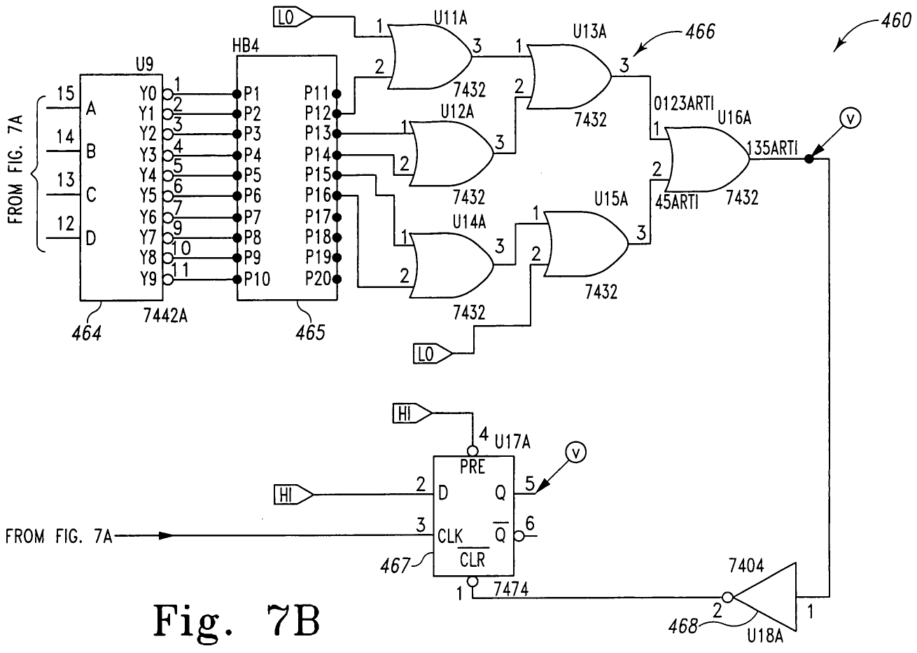

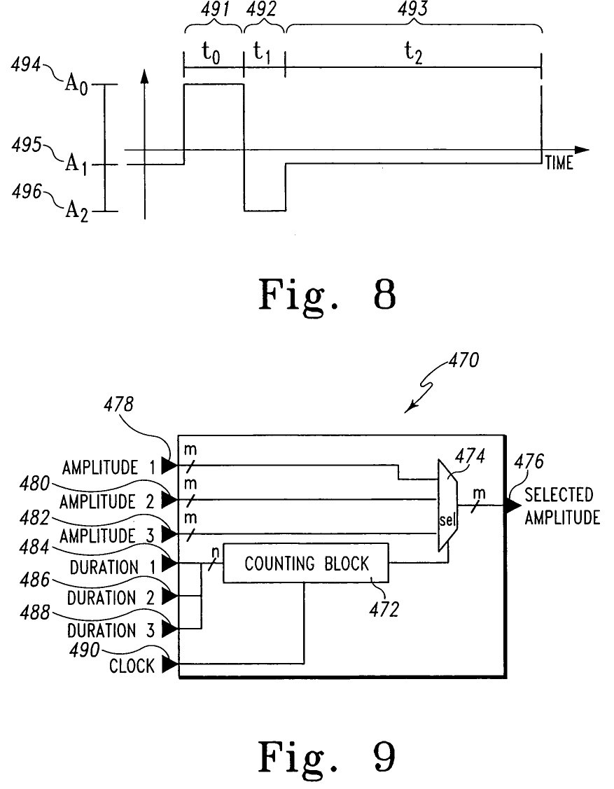

The stimulator module 420 may include a voltage source 422, a pulse generator 426, an inductor 427, a field-converter 428 and the charge storage device 429. A biphasic embodiment 460 of pulse generator 426 implemented using off-the-shelf integrated circuit components, is shown in more detail, for example, in FIGS. 7A-B. In a preferred embodiment, an ASIC is utilized to implement bi-phasic pulse generator 460 which ASIC incorporates the functionality of the illustrated individual integrated circuit components shown in FIGS. 7A-B. A block diagram of a triphasic embodiment 470 of the pulse generator 426 is shown, for example, in FIG. 9. The triphasic pulse generator 470 generates an output signal similar to that shown in FIG. 8. While for power consumption reduction it is preferred that pulse generator 426 create a signal having a polarity that reverses and an on/off duty cycle for each polarity reversal generated, it is within the scope of the disclosure for pulse generator 426 to generate a reversing polarity signal with a 100% on duty cycle.

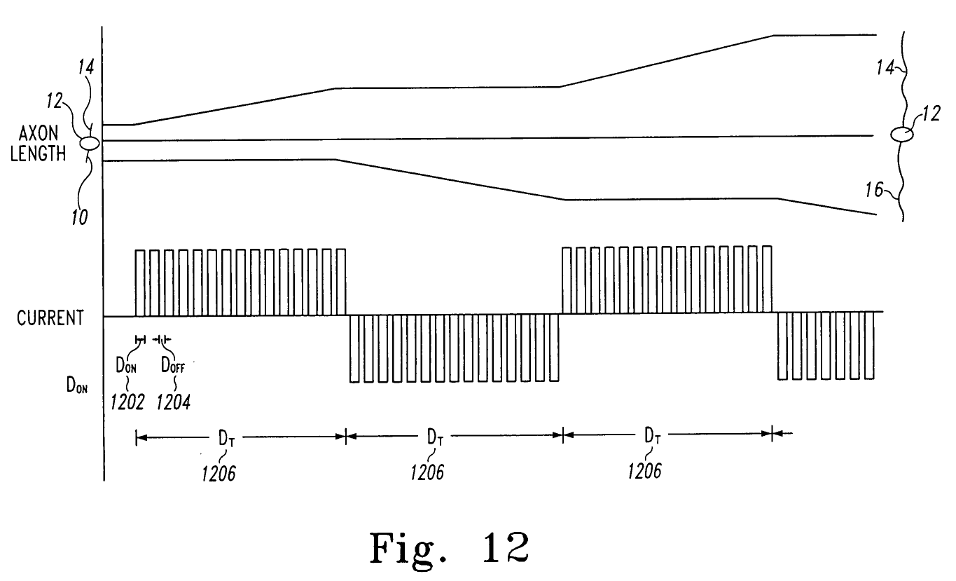

As shown, for example, in FIGS. 7A-B, biphasic pulse generator 460 is implemented using a binary counter 461, a magnitude comparator 462, a buffer 463, a BCD-decimal decoder 464, a second buffer 465, a plurality of input OR gates 466, a NAND gate 468, and a D Flip Flop 467. Such off the shelf integrated circuits are available from many electronic device manufactures. Exemplary part numbers are shown in the drawings. The biphasic pulse generator 460 is configured to receive a plurality of high and low inputs and a fed back clock signal at the binary counter 461. Pulsed signals output by the binary counter 461 are input to the comparator 462 along with various hi and low inputs in the manner illustrated. The biphasic pulse generator 460 outputs a signal such as that shown for example in FIG. 12.

As shown for example, in FIG. 9 a triphasic pulse generator 470 includes a counter block 472, a multiplexer block 474 an output 476, a first amplitude input 478, a second amplitude input 480, a third amplitude input 482, a first duration input 484, a second duration input, 486, a third duration input 488 and a clock input. In one embodiment of the triphasic pulse generator 470 the counting block 472 comprises three counters and the data present at the amplitude inputs 478, 480, 482 and duration inputs 484, 486, 448 comprise six words of data stored in a form of memory (not shown). The three data words present at the duration inputs 484, 486, 488 are illustratively n bits long and represent the duration of each pulse, for example, duration t₀ 491, duration t₁ 492 and duration t₂ 493, as shown, in FIG. 8. The three data words present on the amplitude inputs 478, 480, 482 are illustratively m bits long and represent the amplitudes of each pulse, for example, amplitude A₀ 494, amplitude A₁ 495 and amplitude A₂ 496, as shown, in FIG. 8. In the illustrated embodiment, the three counters in the counting block 472 reset with a value between a value between zero and 2ⁿ−1, where n is the number of bits contained in the counter. This number will represent a time until the counter rolls over. Upon rollover the counter send a flag initiating the next counter. The same operation applies for the second and third counter. While each counter is counting, a multiplexer 474 selects one of the three amplitudes stored in memory determined by the counter currently in operation. Power saving is accomplished by clock gating or reducing the number of counters needed to count the duration of the pulse.

The application of an oscillating electrical field across a lesion and the area adjacent the lesion in the spinal cord of a mammal can stimulate axon growth in both directions, i.e., caudally and rostrally. That is, growth of caudally facing axons will be promoted as will growth of rostrally facing axons. The electrical field is an electrical stimulus which is first applied in one direction or polarity for a predetermined period of time and then applied in the opposite direction or polarity for the predetermined period of time. The polarity of the constant electrical stimulus is reversed after each predetermined period of time.

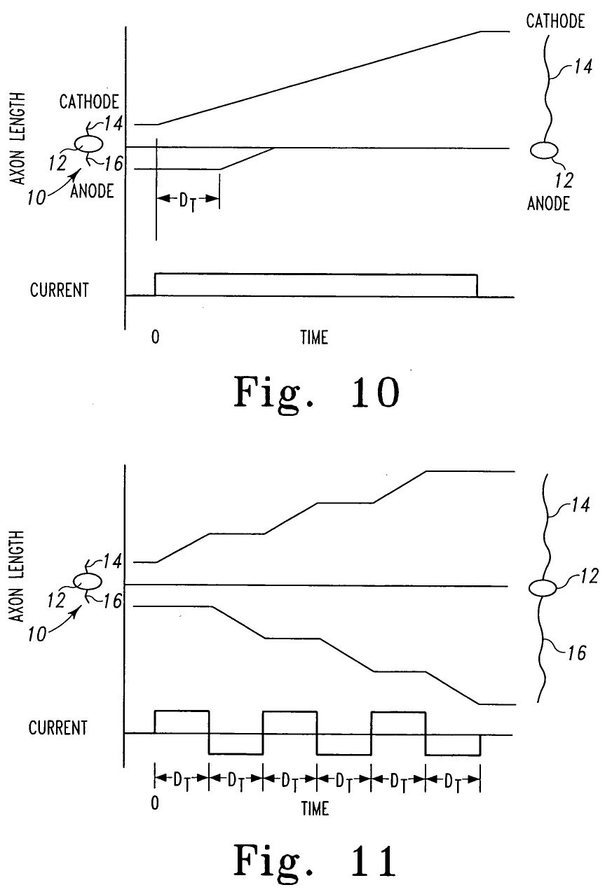

FIGS. 10 and 11 show the effects on axon growth of an applied steady state electrical field (FIG. 10) and by an applied oscillating electrical field (FIG. 11). Referring to FIG. 10, a nerve cell 10 is shown at the left-hand side of FIG. 10 having a cell body or soma 12 from which an axon 14 extends upwardly and an axon 16 extends downwardly. At time 0, a constant electrical stimulus having a first polarity is applied to the nerve cell 10 such that axon 14 will be extending toward the cathode or negative pole of a electrical stimulus signal and axon 16 will be extending toward the anode or positive pole of the electrical stimulus. Axon 14 begins to grow almost immediately. However, after a period of time, i.e., the “die back period” (D_{T}), reabsorption of the anodally facing axon 16 into the cell body 12 begins. Eventually after a sufficient time of continually facing the anode, axon 16 will be completely reabsorbed into cell body 12. At the right-hand side of FIG. 10 for illustration purposes nerve cell 10 is shown wherein axon 14 has grown substantially longer but axon 16 has been reabsorbed into cell body 12.

In FIG. 11, the same reference numbers will be used to identify the elements of FIG. 11 which correspond to elements of FIG. 10. Nerve cell 10 is shown at the left-hand side of FIG. 11 having a cell body 12, an upwardly extending axon 14 and a downwardly extending axon 16. At time 0, a constant electrical stimulus is applied to nerve cell 10 such that axon 14 is extending toward the cathode and axon 16 is extending toward the anode of the electrical stimulus. After a predetermined period of time, the polarity of the electrical stimulus is reversed. Axon 14 will now be extending toward the anode and axon 16 will be extending toward the cathode of the electrical stimulus. The predetermined period of time is selected to be less than the die back period (D_{T}) of the anodal facing axon. Significant die back of anodal facing axons begins to occur about one hour after the electrical stimulus is applied but die back may begin sooner or later. Therefore, the predetermined period should not exceed one hour. As shown in FIG. 11, an oscillating electrical field stimulates growth of the axons facing both direction. This is due to the fact that growth of cathodal facing axons is stimulated almost immediately after the electrical stimulus is applied but die back of the anodal facing axons does not become significant until after the die back period elapses. Since the polarity of the electrical stimulus is switched before the die back period elapses, growth of axons in both directions is stimulated with the result that axons 14, 16 of nerve cell 12 both grow significantly longer as shown at the right-hand side of FIG. 11.

In accordance with the present disclosure, the nerves in the central nervous system of a mammal are stimulated to regenerate by applying an oscillating electrical field to the central nervous system. The oscillating electrical field is a voltage potential stimulus which is first applied in one direction for a predetermined period of time, and then applied in the opposite direction for the predetermined period of time. In other words, the polarity of the voltage potential stimulus is reversed after each predetermined period of time. The predetermined period of time is selected to be less than the die back period of anodal facing axons, but long enough to stimulate growth of cathodal facing axons. This predetermined period will be termed the “polarity reversal period” of the oscillating electrical field. In one disclosed embodiment, this polarity reversal period is between about thirty seconds and about sixty minutes. According to at least one embodiment of the present disclosure, there may be a period between each polarity reversal period where no voltage potential stimulus is applied (an “off cycle”). According to at least one embodiment of the present disclosure, two or more consecutive polarity reversal periods may be followed by an off cycle.

Circuit 400 when implemented with a biphasic pulse generator 460 (FIGS. 7A-B), triphasic pulse generator 480 (FIG. 9) or other multi-phasic pulse generator as the pulse generator 426 comprises a chopping circuit. The voltage potential difference and thus the electrical field between the electrode of the first and second implant 102, 104 is “chopped” or turn off for a short but fixed amount of time. For example, by setting jumper 620 to a 25% duty cycle and jumper 622 to a 50% duty cycle, the electrical field exhibits an on duty cycle Don 1202 of 75% (jumper 620 plus jumper 622) and off duty cycle Doff 1204 for 25% of the time, chopped once per minute producing a wave form as shown in FIG. 12. If this amount of time is small enough compared to the overall time, the nerve cell regeneration continues at the same rate as if the electrical field were held steady. However, chopping the electrical field in the manner illustrated increases battery life, or enables the battery to power other device functions while maintaining a lifespan sufficient for regeneration to be substantially completed. Additionally, punctuated, pulsatile or discontinuous oscillating electric fields are believed to work as well, if not, in some case when utilized to heal certain types of nerves, better than, constant oscillating electric fields. Thus, there is the expectation that the chopping circuit will generate a pulsatile electric field that may improve. functional recovery as well as save battery life.

In one disclosed embodiment, where polarity reversal period DT 1206 of the oscillating electrical field is set to 10 minutes and the duty cycle of the electrical field is set to 75%, circuit 400 produces an output wave form as shown in FIG. 12. It is within the scope of the disclosure for the polarity reversal period to be between about thirty seconds and about sixty minutes. It is also within the scope of the disclosure for the polarity reversal period to be between a minimal clinically effective value to stimulate nerve regeneration in the cathode-facing axon and a value less than the beginning of the die-back period in the anode-facing axon. Clinically effective results can readily be obtained when the reversal period is set between ten and twenty minutes. Highly effective clinical results may be achieved with the duty cycle set to approximately fifteen minutes. It is also within the scope of the disclosure, though not preferred because regeneration of axons induced to die back through the area of die back will be required before therapeutic growth will be induced, for the polarity reversal period to exceed the beginning of the die back period but be less than the time for die back to proceed to the point of killing the nerve cell.

It is within the scope of the disclosure for the on duty cycle 1202 to be between 60% and 99%. Clinically effective results may be obtained in one embodiment when the on duty cycle 1202 is between 70% and 85%. Clinically effective results may be obtained in another embodiment when the on duty cycle 1202 is between 75% and 80%.

According to at least one embodiment of the present disclosure employing a pulsatile field, there may be an off cycle between each polarity reversal period, or there may be two or more consecutive polarity reversal periods followed by an off cycle.

In operation, a first implant 102 and a second implant 104 each comprising circuit 400 is implanted into an injured mammal shortly after the time of central nervous system injury. The implants 102, 104 remain implanted for a period of time post-injury. For example, the implants 102, 104 remain implanted for up to fourteen weeks in humans.

Power is applied to the implants 102, 104 during implantation. When power is applied, the circuit generates an oscillating electrical field between the electrode 108 of implant 102 and the electrode 108 of implant 104. That is, the circuit generates an electrical field the polarity of which is reversed periodically after the expiration of a predetermined period of time determined by the operation of the pulse generator 426. The electrode of implant 102 and the electrode of implant 104 alternately comprise cathode and anode terminals, respectively, depending upon the polarity of the stimulus.

The voltage potential difference between the electrode 1089 of implant 102 and the electrode 108 of implant 104 is selected to provide sufficient field strength in the section of the spinal cord in which nerve regeneration is to be stimulated. A field strength of 200 μV/mm in the spinal cord adjacent the lesion will stimulate regeneration. The potential difference needed to achieve this field strength is determined by the geometry of the animal in which the implants 102, 104 are used and the location of the nearest electrode 108 to the lesion. While a field strength of 200 μV/mm will stimulate regeneration, a field strength of 600 μV/mm has been found to produce clinically effective nerve regeneration in other devices.

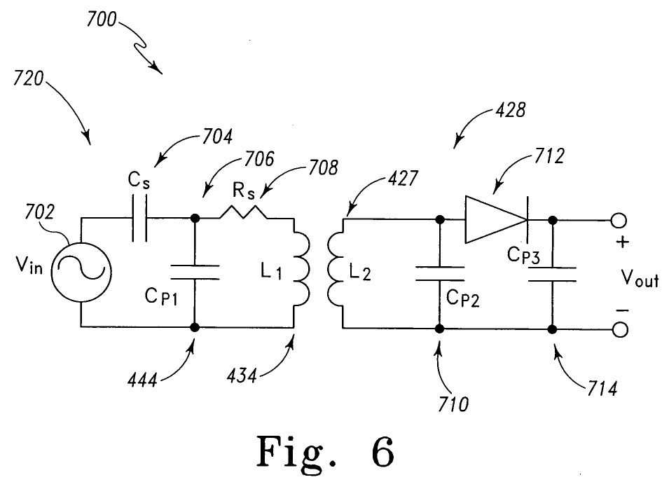

The inductors 427 and 434 and other power coupling components are shown in more detail in FIG. 6. The field-converter 428 may be a radio frequency field converter. The stimulator module 420 may communicate via the wireless data module 410 with the external module 430 via antennas 418 and 432, respectively. The external module 430 may also include a subcutaneous charging device 444 for inductively charging the charge storage device 429 via field converter 428.

In operation, the wireless data module 410 receives power from stimulation module 420 that receives power from the external module 430, stores the power for a time in charge storage device 429, and uses the stored power to generate a field between the electrode 440 of electronic implant 102 and the electrode 440 of electronic implant 104. The electromagnetic power coupling circuit 700, shown in FIG. 6, shows the field converter 428 in more detail. Additionally, the external portion 720 of the power coupling circuit 700 or the subcutaneous charging device 444 is also shown in FIG. 6. A voltage source 702 of the external portion 720 is coupled to an R-L-C circuit comprising first and second capacitors 704 and 706, a resistor 708, and an inductor 434. The external portion 720 generates an electromagnetic field, which may be induced into the inductor 427 of the field converter 428 when the inductors 434 and 427 are in proximity to one another. When that occurs, the inductor 427 provides an AC voltage to the simple rectifier circuit comprising first and second capacitors 710 and 714, and diode 712. In this manner, the field converter 428 may operate to transform coupled fields to direct current fields through charge-rectifying and/or signal conditioning. The field converter 428 may also regulate coupled power delivery for appropriate charging of the charge storage device 429.

Transcutaneous recharging of the charge storage device 429 can be accomplished using medically approved voltage sources such as the Quallion QL100E (weight 4 grams; capacity, 100 mAh; Operating Voltage 2.7-4.2 V; size 14.5 mm by 15.6 mm). The largest component of the circuit 400 determining its overall size is the size of the charge storage device 429. Thus, decreasing the size of the device by using a rechargeable unit for the charge storage device 429 may reduce the size of the circuit 400 to sixty percent or smaller of prior art devices. This decrease in size may simplify surgical implantation, and the time of implantation of the electronic implants 102, 104. Other medical issues, such as contact necrosis, also vary with the size of the circuit 400 which in turn dictates to some extent the size of the case 118 of each electronic implant 102, 104. The timing of recharging cycles will depend on the programmed stimulation parameters. However, charging could be accomplished at night while the patient is asleep, or for shorter periods during the day.

Since the circuit 400 may be located rather superficially in back musculature beneath the back skin, an additional pair of redundant recharging electrodes may be left in situ next to the circuit 400. These redundant recharging electrodes may be externalized simply by. use of a local anesthetic and simple approach through the skin. Under special or unforeseen situations, the circuit 400 can be recharged directly by attachment of these two electrodes to a hardwired recharging unit.

While this invention has been described as having a preferred design, the present invention can be further modified within the scope and spirit of this disclosure. This application is therefore intended to cover any variations, uses, or adaptations of the invention using its general principles. For example, the methods disclosed herein and in the appended claims represent one possible sequence of performing the steps thereof. A practitioner of the present invention may determine in a particular implementation of the present invention that multiple steps of one or more of the disclosed methods may be combinable, or that a different sequence of steps may be employed to accomplish the same results. Each such implementation falls within the scope of the present invention as disclosed herein and in the appended claims. Furthermore, this application is intended to cover such departures from the present disclosure as come within known or customary practice in the art to which this invention pertains and which fall within the limits of the appended claims.

What is claimed is:

1. A neural injury treatment device (100) comprising:
a first electronic implant (102) configured to generate a first potential difference relative to a body of a patient; and
a second electronic implant (104) configured to generate a second potential difference relative to the body of the patient, said second potential having a polarity opposite the polarity of the first potential difference, said second electronic implant (104) being configured to be wirelessly communicatively coupled and electrically coupled to the first electronic implant (102) when spaced apart therefrom in the body of a patient;
wherein said first electronic implant (102) and said second electronic implant (104) are configured to change their polarities substantially simultaneously.

2. The device (100) of claim 1 further comprising an external controller (430) in wireless communication with the first electronic implant 102.

3. The device of claim 2 wherein the first electronic implant (102) and second electronic implant (104) are in a master slave configuration, the first electronic implant acting as the master to establish a polarity of an electrode (440) of the first electronic implant (102) and the second electronic implant (104) being configure to establish an opposite polarity for an electrode (440) of the second electronic implant (102).

4. The device of claim 2 wherein the second electronic implant (104) is in wireless communication with the external controller (430).

5. The device of claim 1 wherein the first and second electronic implants 102, 104 each comprise a transcutaneously rechargeable power supply (429).

6. The device of claim 1 wherein the first and second electronic implants 102, 104 each including a ceramic skin encompassing an electrode (440) and electronic circuitry (410, 420) facilitating wireless communication.

7. The device of claim 6 wherein the electronic circuitry (410, 420) includes an antenna 418 comprising a planar microstrip antenna.

8. The device of claim 6 wherein the electronic circuitry (410, 420) includes an antenna (418) comprising a monolithic microwave integrated-circuit (MMIC) radiating structure.

9. The device of claim 6 wherein the electronic circuitry (410, 420) comprises microelectronic circuitry.

10. The device of claim 6 wherein the first and second electronic implants (102, 104) each comprise a ground electrode (112) extending beyond the ceramic skin (110).

11. The device of claim 1 wherein the first electronic implant (102) is configured to facilitate insertion of the first electronic implant into a mammalian body utilizing minimally invasive surgical techniques.

12. The device of claim 11 wherein the first electronic implant (102) is configured to be slidably received within a lumen of a trochanter (300).

13. The device of claim 10 wherein the first electronic implant is configured to include a suture tab (114) electrically coupled to the ground electrode (112).

14. An apparatus for stimulating axon (14, 16) growth of the nerve cells (12) in the spinal cord of mammals, the apparatus comprising:
a first electronic implant (102) having an electrode (440), a voltage generating circuit (420) to create a voltage potential difference between the electrode (440) and the mammal and a polarity reversing circuit electrically coupled to the voltage generating circuit and configured to reverse the polarity of the voltage potential difference between the electrode (440) and the body of the mammal each time a predetermined period of time elapses; and
a second electronic implant (104) having an electrode (440), a voltage generating circuit to create a voltage potential difference between the electrode and the mammal and a polarity reversing circuit electrically coupled to the voltage generating circuit and configured to reverse the polarity of the voltage potential difference between the electrode (440) and the body of the mammal each time a predetermined period of time elapses, the second electronic implant being communicatively coupled to first electronic implant when spaced apart therefrom;
wherein said first electronic implant (102) and said second electronic implant (104) are configured to change their polarities substantially simultaneously.
